(19) United States
(12) Patent Application Publication
(10) Pub. No.: US 2008/0242877 A1
(43) Pub. Date: Oct. 2, 2008

(54) Intermediates and processes for the synthesis of Ramelteon

(76) Inventors: Vinod Kumar Kansal, Haryana (IN); Dhirenkumar N. Mistry, Gujarat (IN); Sanjay L. Vasoya, Gujarat (IN)

Correspondence Address: KENYON & KENYON LLP, ONE BROADWAY, NEW YORK, NY, 10004, US

(21) Appl. No.: 12/072,518

(22) Filed: Feb. 26, 2008

Related U.S. Application Data
(60) Provisional application No. 60/903,782, filed on Feb. 26, 2007.

(51) Int. Cl. C07D 307/93; C07C 69/618; C07C 233/01

(52) U.S. Cl. 549/458; 560/56; 564/180

(57) ABSTRACT

Provided are intermediates and processes for preparation of Ramelteon.

### CROSS REFERENCE

The Present application claims benefit of U.S. provisional Application No. 60/903,782, filed Feb. 26, 2007, whose entire disclosure is incorporated herein by reference.

### FIELD OF THE INVENTION

The present invention relates to synthesis of (S)—N-[2-(1, 6, 7, 8-tetrahydro-2H-indeno-[5,4-b]furan-8-yl)ethyl]propionamide i.e. Ramelteon.

### BACKGROUND OF THE INVENTION

ROZEREM® (Ramelteon) is a melatonin receptor agonist with both high affinity for melatonin MT1 and MT2 receptors and selectivity over the MT3 receptor. The empirical formula for Ramelteon is C₁₆H₂₁NO₂, and its molecular weight is 259.34. Ramelteon is freely soluble in methanol, ethanol, dimethylsulfoxide (DMSO), 1-octanol and is highly soluble in water and aq. buffer. Ramelteon has the following chemical structure:

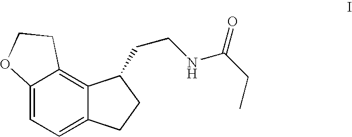

Ramelteon is the active ingredient and sold under the brand name of ROZEREM®. Ramelteon is approved by the United States Food and Drug Administration for the treatment of insomnia characterized by difficulty with sleep onset.

Different processes for preparing (S)—N-[2-(1,6,7,8-tetrahydro-2H-indeno-[5,4-b]furan-8-yl)ethyl]propionamide i.e. Ramelteon are disclosed in U.S. Pat. No. 6,034,239, JP 11080106, JP 11140073 and WO 2006/030739.

U.S. Pat. No. 6,034,239 discloses the following processes for the preparation of Ramelteon:

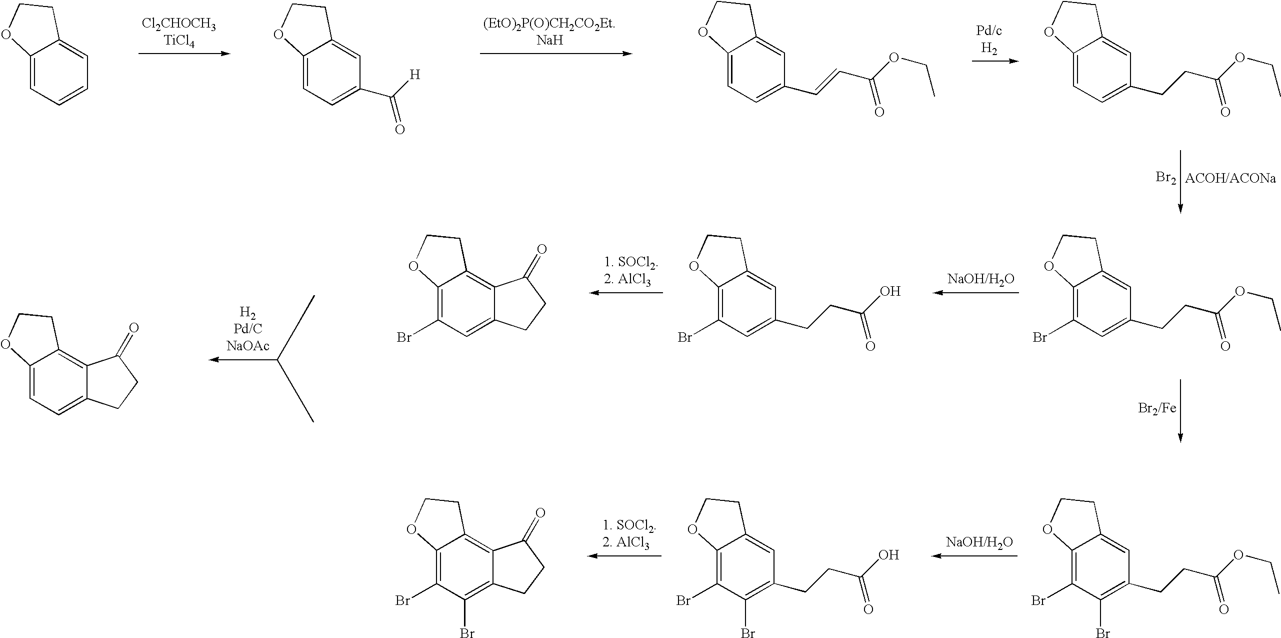

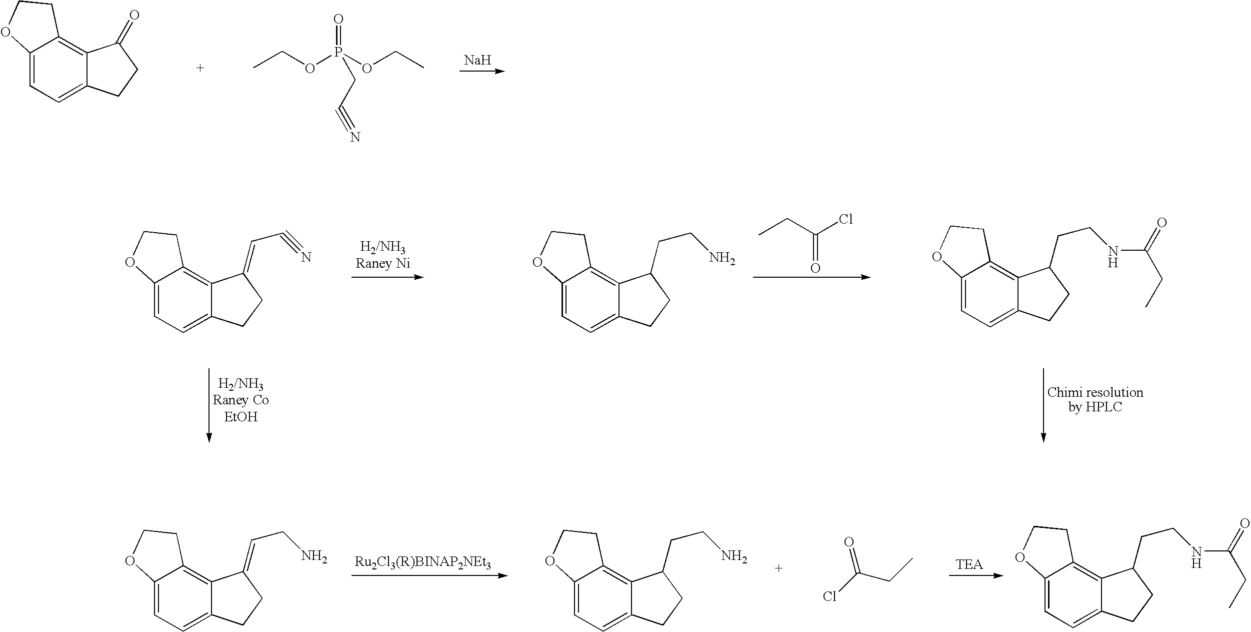

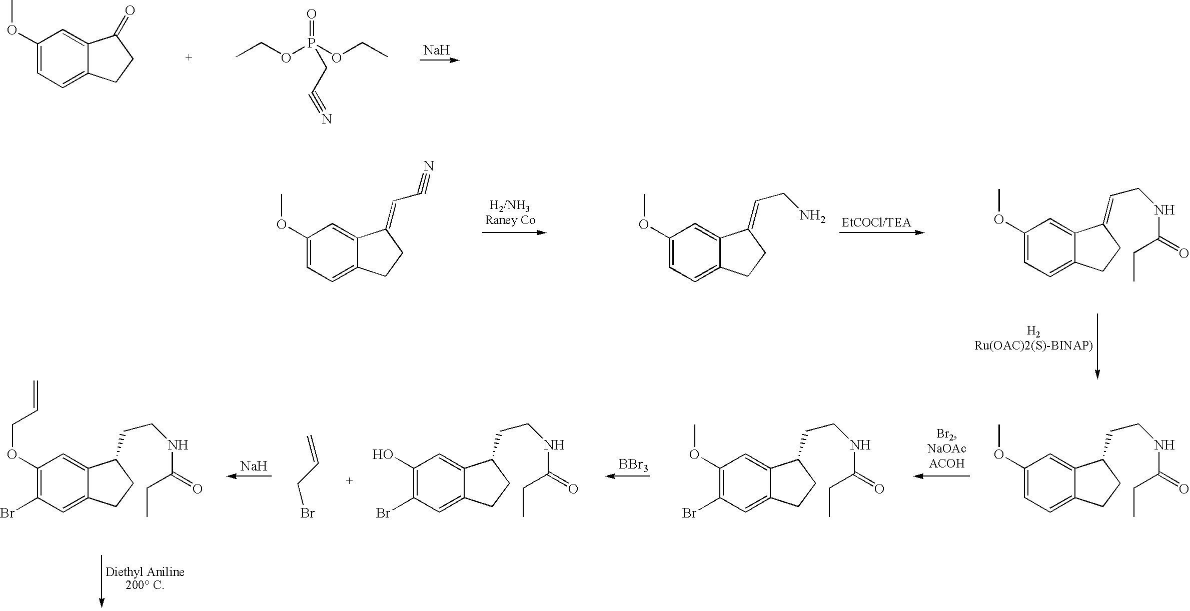

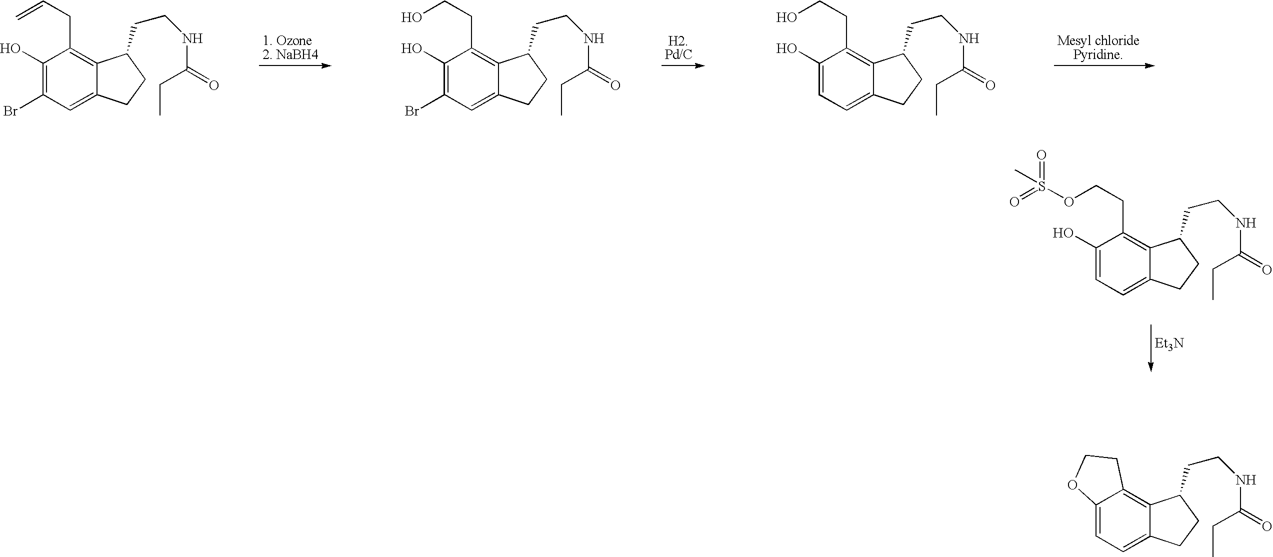

Japan Patent Publication No. 11080106 discloses the following processes for the preparation of Ramelteon:

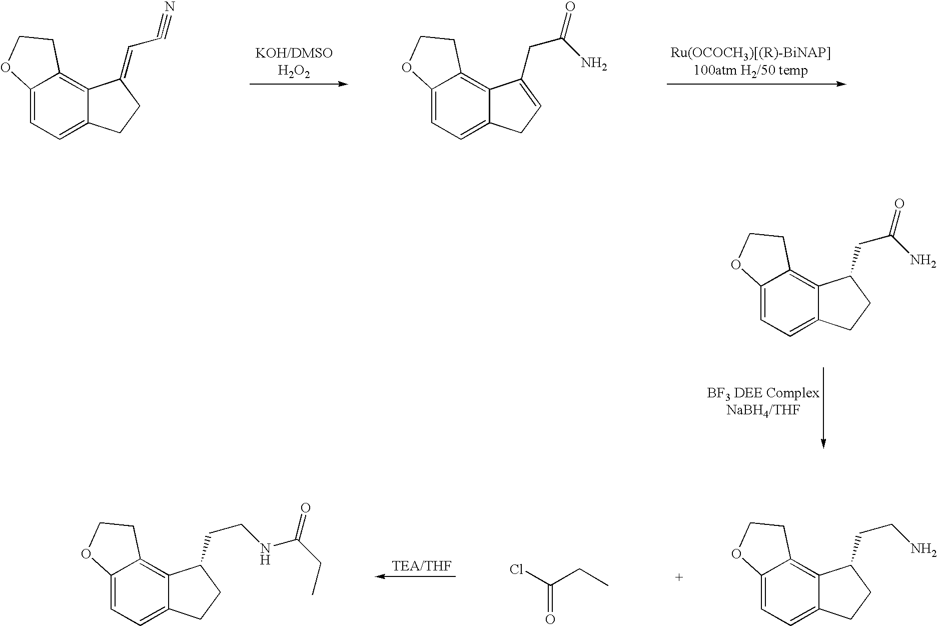

Japan Patent Publication No. 11140073 discloses the following processes for the preparation of an intermediate of Ramelteon:

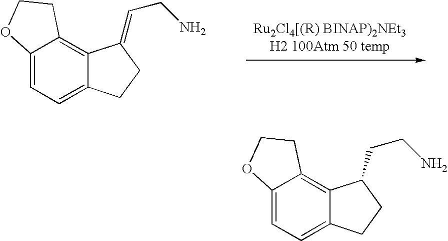

PCT Publication No. 2006/030739 discloses the following processes for the preparation of an intermediate of Ramelteon:

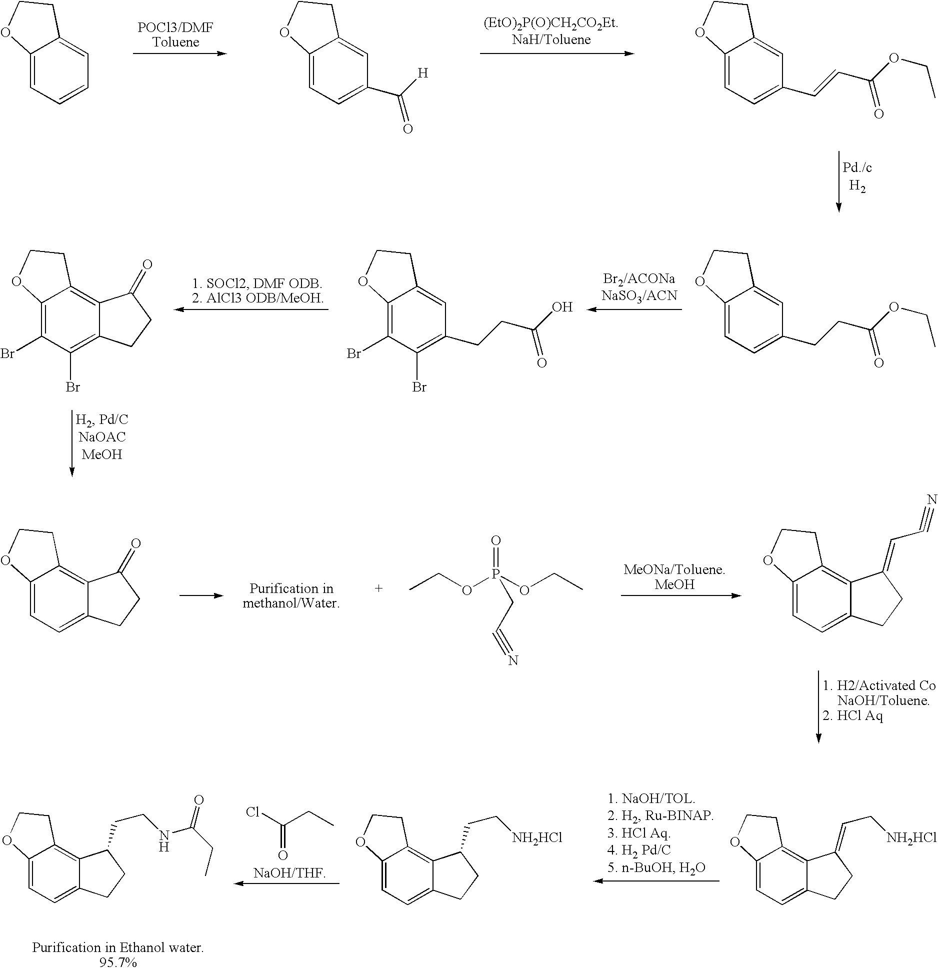

The present invention provides additional processes for preparation Ramelteon and intermediates thereof.

### SUMMARY OF INVENTION

In one embodiment, the present invention provides a process for producing Ramelteon intermediate of formula IV, comprising the step of: combining the compound of Formula II with compound of formula III in the presence of base and organic solvent:

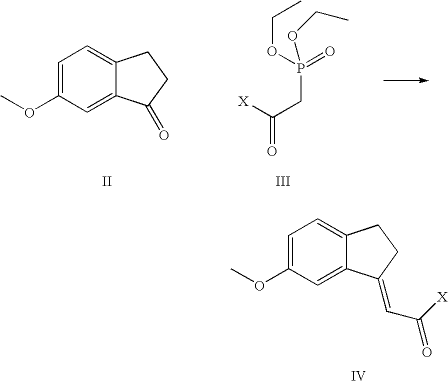

### Wherein

X=O-Alkyl or —NH₂. In one embodiment X is ethoxy.

In one embodiment, the present invention encompasses a process for preparing Ramelteon, by preparing the compound of Formula IV as described above, and converting it to Ramelteon.

In another embodiment, the present invention encompasses a process for preparing a Ramelteon intermediate of formula V, comprising chiral reduction of compound of formula IV in presence of Ru-BINAP complex under hydrogen atmosphere in an organic solvent:

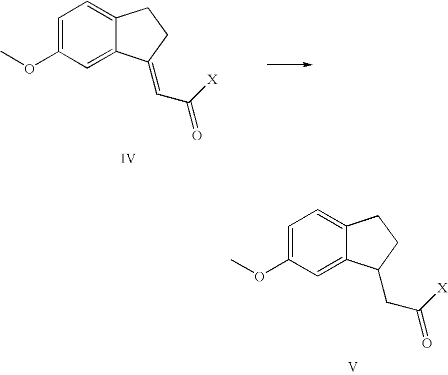

### Wherein

X=O-Alkyl or —NH₂. In one embodiment X is ethoxy.

In one embodiment, the present invention encompasses a process for preparing Ramelteon, by preparing the compound of Formula V as described above, and converting it to Ramelteon.

In another embodiment, the present invention encompasses a process for preparing a Ramelteon intermediate of formula VI, comprising reacting the compound of formula V with brominating agent in presence of an acid or alkaline salt of an acid:

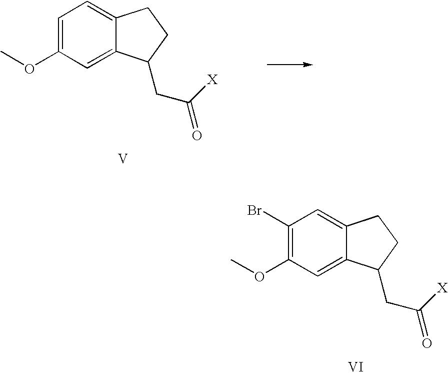

### Wherein

X=O-Alkyl or —NH₂. In one embodiment X is ethoxy.

In one embodiment, the present invention encompasses a process for preparing Ramelteon, by preparing the compound of Formula VI as described above, and converting it to Ramelteon.

In another embodiment, the present invention encompasses a process for preparing a Ramelteon intermediate of formula VII, comprising removing the protective group for the hydroxyl group in compound of formula VI:

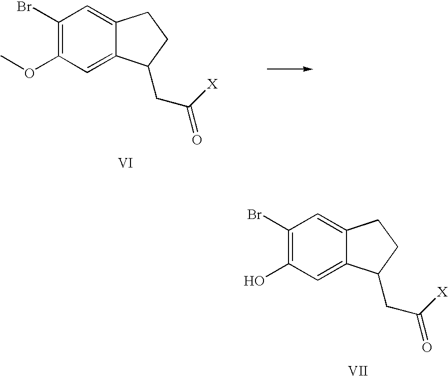

Wherein X=O-Alkyl or —NH₂. In one embodiment X is ethoxy.

The deprotection of hydroxyl group can be carried out by a reagent selected from the group comprising of boron tribromide (BBr₃), hydrobromic acid (HBr) in acetic acid, pyridine-HBr, quarternary ammonium salt, 2-(diethylamino) ethanethial.HCl, trifluoroacetic acid, anisole and aluminum trichloride (AlCl₃). The reaction is conducted in a solvent, for example, halogenated hydrocarbons, a C₆ to C₁₄ aromatic hydrocarbon, a C₁ to C₇ aliphatic hydrocarbon, a C₁ to C₅ alcohol, a C₂ to C₇ ester, and a C₂ to C₇ ether, a C₁ to C₇ organic acid, inorganic acid or a suitable mixture thereof.

In one embodiment, the present invention encompasses a process for preparing Ramelteon, by preparing the compound of formula VII as described above, and converting it to Ramelteon.

In another embodiment, the present invention encompasses a process for preparing the Ramelteon intermediate of formula IX, comprising reacting the compound formula VII with the compound of formula VIII in presence of a base to produce the compound of formula IX:

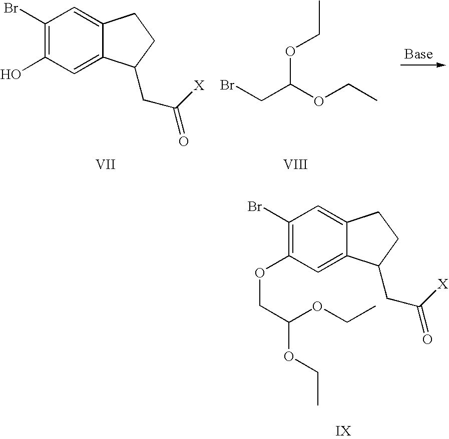

Wherein X=O-Alkyl or —NH₂. In one embodiment X is ethoxy.

In one embodiment, the present invention encompasses a process for preparing Ramelteon, by preparing the compound of formula IX as described above, and converting it to Ramelteon.

In another embodiment, the present invention encompasses a process for preparing the Ramelteon intermediate of formula X, cyclizing the compound of formula IX to produce compound of formula X:

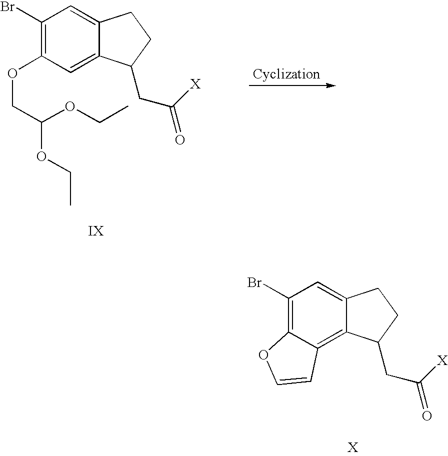

Wherein X=O-Alkyl or —NH₂. In one embodiment X is ethoxy.

In one embodiment, the present invention encompasses a process for preparing Ramelteon, by preparing the compound of Formula X as described above, and converting it to Ramelteon.

In another embodiment, the present invention encompasses a process for preparing the Ramelteon intermediate of formula XI, comprising de-bromination of compound of formula X by reduction:

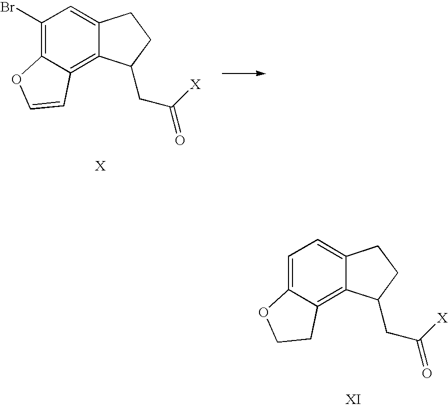

Wherein X=O-Alkyl or —NH₂. In one embodiment X is ethoxy.

In one embodiment, the present invention encompasses a process for preparing Ramelteon, by preparing the compound of Formula XI as described above, and converting it to Ramelteon.

In another embodiment, the present invention encompasses a process for preparing the Ramelteon intermediate of formula XII, comprising reacting the compound of formula XI with an aminating agent:

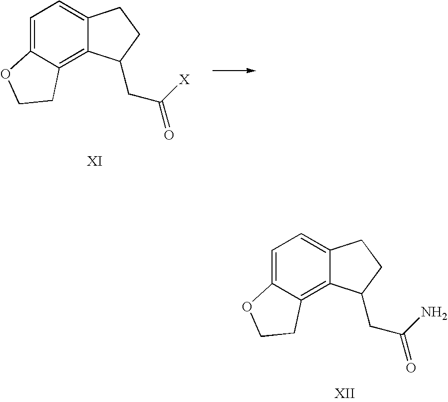

Wherein X=O-Alkyl or —NH₂. In one embodiment X is ethoxy.

In one embodiment, the present invention encompasses a process for preparing Ramelteon, by preparing the compound of Formula XII as described above, and converting it to Ramelteon.

In another embodiment, the present invention encompasses a process for preparing a Ramelteon intermediate of formula XIII, comprising reduction of compound of formula XII with a reducing agent:

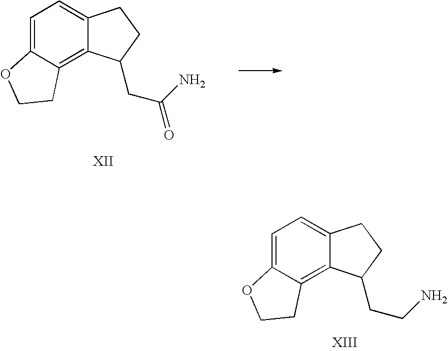

In another embodiment, the present invention encompasses a process for preparing Ramelteon of formula I, comprising reacting compound of formula XIII with propionyl chloride and base to produce Ramelteon of formula I.

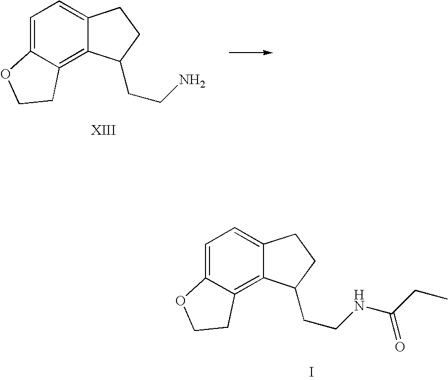

In another embodiment, the present invention encompasses a process for preparing Ramelteon (with mild conditions) of Formula I comprising:

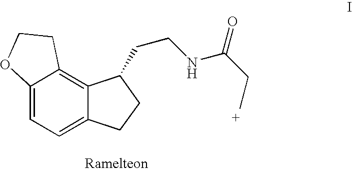

(a) reacting the compound of formula II with compound of formula III in presence of an inorganic base and an organic solvent:

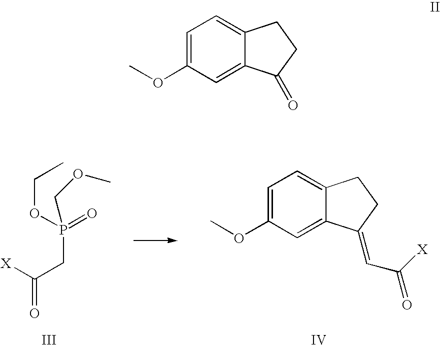

(b) chiral reduction of compound of formula IV to obtain compound of formula V:

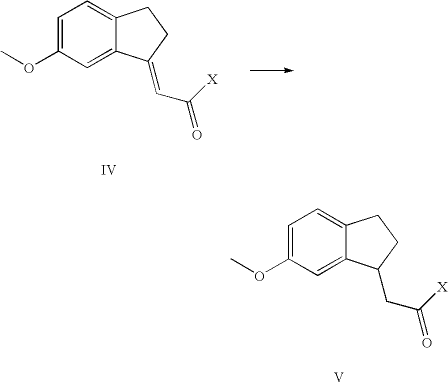

(c) combining compound of formula V with brominating agent in presence of an acid or alkaline salt of an acid:

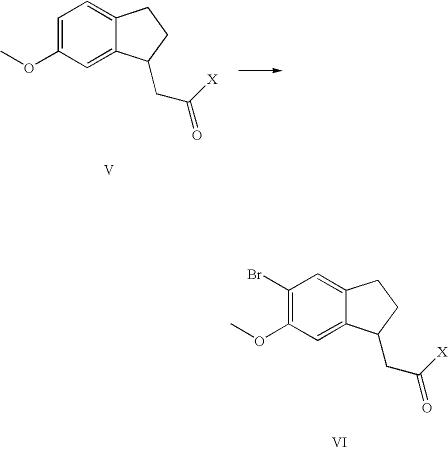

(d) demethylation of the compound of formula VI to obtain the compound of formula VII in the presence of demethylating reagent such as BBr3, HBr in acetic acid, pyridine-HBr, quarternary ammonium salt, 2-(diethylamino) ethanethiol.HCl, trifluoroacetic acid, anisole and AlCl₃ and an organic solvent:

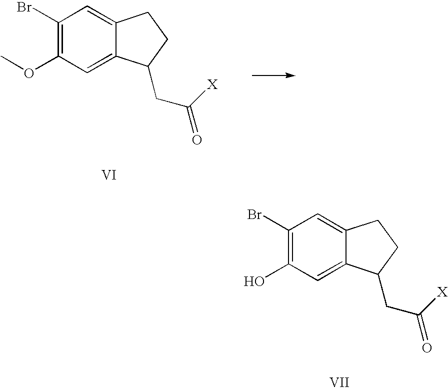

(e) combining the compound of formula VII with compound of formula VIII in presence of base to produce the compound of formula IX:

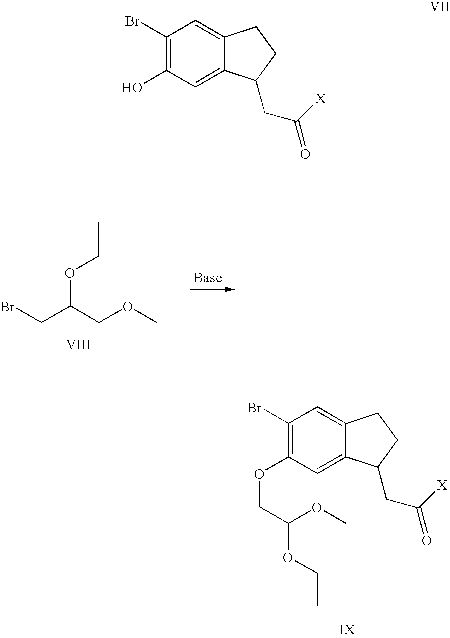

(f) cyclizing the compound of formula IX in presence of methane sulfonic acid, trifluoroacetic acid, p-toluene sulphonic acid to obtain the compound of formula X:

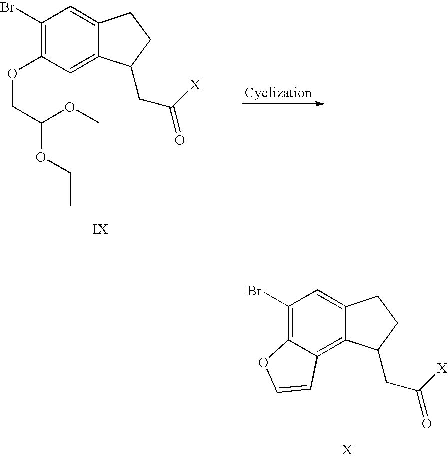

(g) debrominating the compound formula X by reacting the compound of formula X with dehalogenation reaction to produce the compound of formula XI:

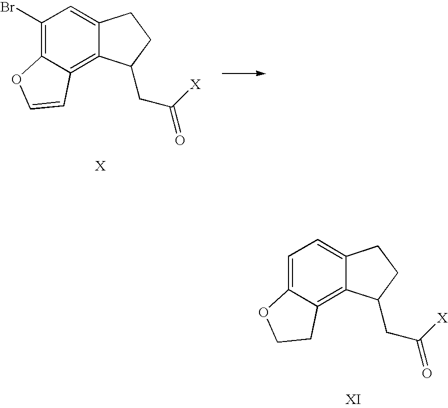

(h) combining the compound of formula XI with an aminating agent to obtain the compound of formula XII:

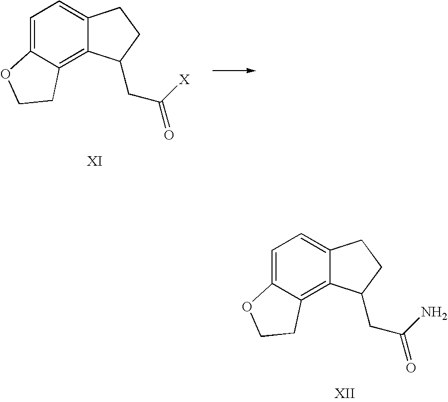

(i) reducing the compound formula XII with, boron trihalide complex, and sodium borohydride to produce the compound of formula XIII:

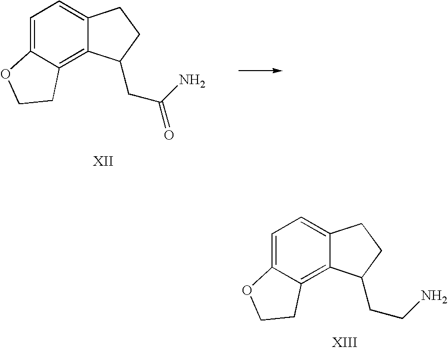

(j) combining the compound of formula XIII with propionyl chloride and base to produce Ramelteon of formula I:

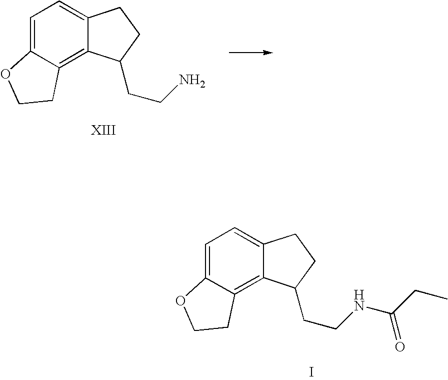

Wherein X=O-Alkyl or —NH₂. In one embodiment X is ethoxy.

### DETAILED DESCRIPTION OF THE INVENTION

As used herein, the term ‘alkyl’ refers to a straight or branched hydrocarbon chain radical consisting of carbon and hydrogen atoms, containing no unsaturation, having from one to eight carbon atoms, and which is attached to the rest of the molecule by a single bond, e.g., methyl, ethyl, n-propyl, 1-methylethyl (isopropyl), n-butyl, n-pentyl, 1,1-dimethylethyl (t-butyl), and the like.

As used herein, the term “aryl” refers to aromatic radicals having in the range of 6 up to 14 carbon atoms such as phenyl, substituted phenyl, naphthyl, tetrahydronapthyl, indanyl, biphenyl and the like.

As used herein, the term “arylalkyl” refers to an aryl group as defined above directly bonded to an alkyl group as defined above. e. g., —CH₂C₆H₅, —C₂H₄C₆H₅ and the like.

As used herein, the term “alkoxy” denotes alkyl group as defined above attached via oxygen linkage to the rest of the molecule. Representative examples of those groups are —OCH₃, —OC₂H₅ and the like.

As used herein, the term “alkoxycarbonyl” denotes —C(O)— is linked to alkoxy group such —C(O)OCH₃, —C(O)OC₂H₅ etc. The term “alkoxy” is defined as above.

As used herein, the term “cycloalkyl” denotes a non-aromatic mono or multicyclic ring system of about 3 to 12 carbon atoms such as cyclopropyl, cyclobutyl, cyclopentyl, cyclohexyl and examples of multicyclic cycloalkyl groups include perhydronapththyl, adamantyl and norbornyl groups bridged cyclic group or sprirobicyclic groups e.g. sprio (4,4) non-2-yl.

The substituents in the ‘substituted alkyl’, ‘substituted aryl, ‘substituted arylalkyl’ and substituted alkoxycarbonyl and may be the same or different which one or more selected from the groups such as hydrogen, hydroxy, carboxyl, substituted or unsubstituted alkyl, substituted or unsubstituted alkoxy, substituted or unsubstituted aryl, substituted or unsubstituted arylalkyl, substituted or unsubstituted cycloalkyl, substituted or unsubstituted amino;

The term “amine” refers to —NH₂.

The substituents in the ‘substituted alkyl’, ‘substituted aryl, ‘substituted arylalkyl’ and substituted alkoxycarbonyl and may be the same or different which one or more selected from the groups such as hydrogen, hydroxy, carboxyl, substituted or unsubstituted alkyl, substituted or unsubstituted alkoxy, substituted or unsubstituted aryl, substituted or unsubstituted arylalkyl, substituted or unsubstituted cycloalkyl, substituted or unsubstituted amino.

As used herein, aqueous ammonia refers to 5-35% aqueous ammonia.

As used herein, the term “halogenated hydrocarbons” refers to cyclic or acyclic, saturated or unsaturated aliphatic or aromatic hydrocarbons. Examples of halogenated hydrocarbons include, but are not limited to, halogenated alkanes such as chloromethane, dichloromethane, chloroethane, dichlorotrifluoroethane, difluoroethane, hexachloroethane, pentafluoroethane, halogenated alkenes such as such as tetrachloroethene, dichloroethene, trichloroethene, vinyl chloride, chloro-1,3-butadiene, chlorotrifluoroethylene, or halogenated benzenes such as benzotrichloride, benzyl chloride, bromobenzene, chlorobenzene, chlorotoluene, dichlorobenzene, fluorobenzene, or trichlorobenzene. The preferred halogen is chlorine. The preferred halogenated hydrocarbons are aromatic hydrocarbons or C1-C4 alkanes, and more preferably chlorinated aromatic hydrocarbons or C1-C4 alkanes. The more preferred halogenated hydrocarbons are chlorobenzene, o- or p-dichlorobenzene, dichloromethane, or o-chlorotoluene.

In one embodiment, the present invention provides a process for producing Ramelteon intermediate of formula IV, comprising the step of: combining the compound of formula II with compound of formula III in the presence of a base and an organic solvent:

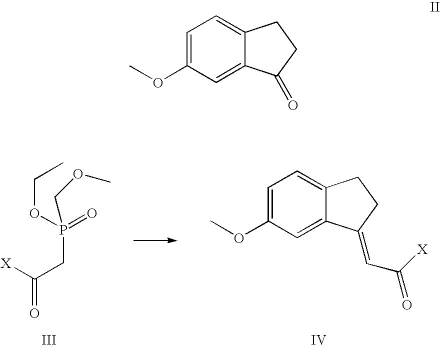

Wherein X=O-Alkyl or —NH₂. In one embodiment X is ethoxy.

Suitable bases include alkali metal carbonates, hydroxides or hydrides, for example potassium bicarbonate, sodium bicarbonate, potassium carbonate, sodium carbonate, sodium hydroxide, potassium hydroxide, sodium hydride, and potassium hydride; organic bases of the structure NR₃ wherein R is an organic radical of 1-5 carbons, like triethyl amine, diisopropyl ethyl amine, N-methyl morpholine; metal amides, for example, sodium amide, lithium diisopropylamide, lithium hexamethyldisilazide, metal alkoxides, for example, sodium methoxide, sodium ethoxide potassium tert-butoxide, etc. n-butyl lithium (n-BuLi); 1,8-diazabicyclo (5.4.0) undec-7-ene; hexamethylphosphoramide. Sodium hydride (NaH) is a preferred base for use in the practice of the present invention.

Suitable organic solvents can be selected from the group consisting of C₆₋₁₀ substituted aromatic hydrocarbons, C₁₋₅ aliphatic hydrocarbons, halogenated hydrocarbons, ethers, ketones, esters, nitrites, C₄₋₆ straight, branched or cyclic hydrocarbons, dioxanes, DMF, DMSO, and mixtures thereof. A preferred C₆₋₁₀ substituted aromatic hydrocarbon is either toluene or xylene. The ethers, ketones, esters may be C₂ to C₇. A preferred nitrile is acetonitrile.

The compound of Formula IV can then be used to prepare Ramelteon.

In another embodiment, the present invention encompasses a process for preparing a Ramelteon intermediate of formula V, comprising chiral reduction of compound of formula IV in presence Ru-BINAP complex such as Ru₂Cl₄[(R)-BINAP]₂NEt₃, Ru₂[(R)-BINAP](OAc), Ru(OAc)₂[(R)-T-BINAP], Ru₂Cl₄[(R)-DM-BINAP]₂NEt₃, Ru₂[(R)-T-BINAP](OAc), [RuCl(Benzene)((R)-BINAP)]Cl, [RuCl(p-Cymene)((R)-BINAP)]Cl, [RuBr(p-Cymene)((R)-BINAP)]Br, [RuI(p-Cymene)((R)-BINAP)]I under hydrogen atmosphere in an organic solvent selected from the group consisting of: a C₆ to C₁₄ aromatic hydrocarbon, C₁ to C₅ alcohol, a C₂ to C₇ ester, and a C₂ to C₇ ether, halogenated hydrocarbons or a suitable mixture thereof. Preferable organic solvents are methanol, ethanol, isopropanol (IPA), ethyl acetate, dither ether, diisopropyl ether, dichloromethane, dichloroethane, toluene and xylene. Most preferable solvent is selected from methanol, ethanol and toluene. The hydrogen atmosphere can have a pressure of about 3 to about 5 bar.

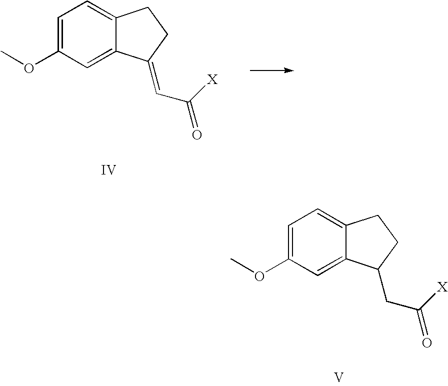

Wherein X=O-Alkyl or —NH₂. In one embodiment X is ethoxy.

The compound of Formula V can then be used to prepare Ramelteon.

In another embodiment, the present invention encompasses a process for preparing the Ramelteon intermediate of formula VI, comprising: reacting the compound of formula V with brominating agent in presence of an acid or an alkaline salt of organic acid or an acid acceptor. The process can be carried out in an organic solvent selected from the group consisting of: a C₆ to C₁₄ aromatic hydrocarbon, a C₁ to C₅ aliphatic hydrocarbon, a C₁ to C₅ alcohol, a C₂ to C₇ ester, and a C₂ to C₇ ether, a C₁-C₇ acid, halogenated hydrocarbons, C₁-C₅ organic acid or a suitable mixture thereof. Preferable solvents are dichloromethane, ethyl acetate, acetonitrile, methanol and acetic acid. Most preferable solvent is methanol or acetic acid. The brominating agent can be used in an amount of 0.2 to 2 moles on the basis of 1 mole of the compound having a structure of chemical formula V.

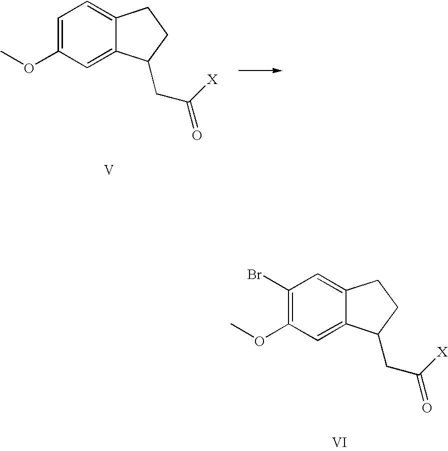

### Wherein X=O-Alkyl, or —NH₂

The brominating agent can be Br₂ or liquid bromine. The acid can include organic or inorganic acid. Preferably, the organic acid is selected from acetic acid, formic acid, methane sulfonic acid, benzoic acid; inorganic acids include hydrochloric acid, hydrobromic acid, phosphoric acid; an alkaline salt of organic acid is selected from Sodium acetate potassium acetate, sodium format. Alkaline salt of organic acid can be selected from the group consisting of sodium acetate, sodium formate, sodium phosphate, potassium acetate, potassium formate and potassium phosphate.

The compound of Formula VI can then be used to prepare Ramelteon.

In another embodiment, the present invention encompasses a process for preparing the Ramelteon intermediate of formula VII, comprising removing the protective group for the hydroxyl group in compound of formula VI.

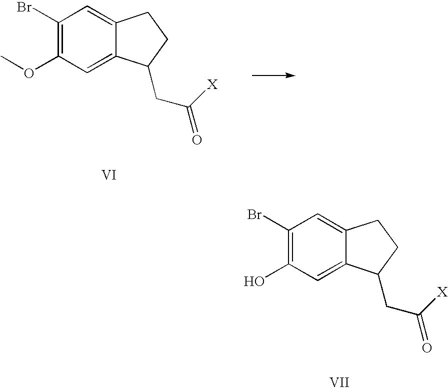

Wherein X=O-Alkyl or —NH₂. In one embodiment X is ethoxy.
The deprotection can be carried out by using reagents such as BBr₃, HBr in acetic acid, pyridine-HBr, quarternary ammonium salt, 2-(diethylamino)-ethanethiol.HCl, trifluoroacetic acid, anisole and AlCl₃. Suitable solvents include halogenated hydrocarbons, a C₆ to C₁₄ aromatic hydrocarbon, a C₁ to C₇ aliphatic hydrocarbon, a C₁ to C₅ alcohol, a C₂ to C₇ ester, and a C₂ to C₇ ether, a C₁ to C₇ organic acid, inorganic acid or a suitable mixture of these solvents. Most preferable solvent is dichloromethane.

The compound of Formula VII can then be used to prepare Ramelteon.

In another embodiment, the present invention encompasses a process for preparing the Ramelteon intermediate of formula IX, comprising reacting the compound formula VII with the compound of formula VIII in presence of a base to produce the compound of formula IX at a temperature of 25-40° C. under nitrogen atmosphere. The condensation reaction further comprises adding an organic solvent. The obtained reaction mixture can be stirred at a temperature of 30-60° C. for 5-8 hr. Typically, the molar amount of compound of formula VIII is 1 to 2 times the molar amount of the compound of formula VII; the molar amount of base will be 1 to 4 times the molar amount of the compound of formula VII;

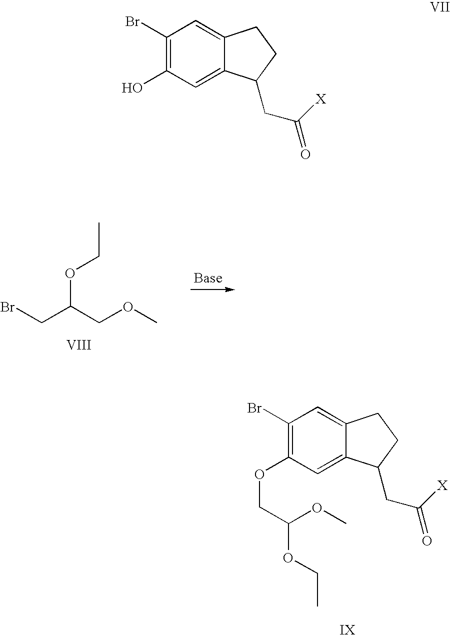

Wherein X=O-Alkyl or —NH₂. In one embodiment X is ethoxy.

The base can be selected from alkali metal carbonates, hydroxides or hydrides, for example potassium bicarbonate, sodium bicarbonate, potassium carbonate, sodium carbonate, sodium hydroxide, potassium hydroxide, sodium hydride, potassium hydride; organic bases like triethylamine (TEA), diethylamine (DEA); metal amides, for example, sodium amide, lithium diisopropylamide, lithium hexamethyldisilazide, etc., metal alkoxides, for example, sodium methoxide, sodium ethoxide potassium tert-butoxide, etc. n-BuLi; 1,8-Diazabicyclo (5.4.0) undec-7-ene; hexamethylphosphoramide etc. potassium hydroxide, and potassium tert-butoxide are preferred bases for use in the practice of the present invention. The organic solvent can be selected from the group consisting of halogenated hydrocarbons, C₆ to C₁₄ aromatic hydrocarbon, C₁ to C₇ aliphatic hydrocarbon, C₁ to C₅ alcohol, C₂ to C₇ ester, C₂ to C₇ ether, DMSO, DMF and mixtures thereof. Preferable organic solvents are isopropyl alcohol, acetone, DMF, DMSO, THF. Most preferable organic solvent is selected from isopropyl alcohol, acetone, and DMF.

The compound of Formula IX can then be used to prepare Ramelteon.

In another embodiment, the present invention encompasses a process for preparing the Ramelteon intermediate of formula X, cyclizing the compound of formula IX to produce compound of formula X. Cyclization can be conducted by, for example, heating the compound, using an acidic substance or a basic substance.

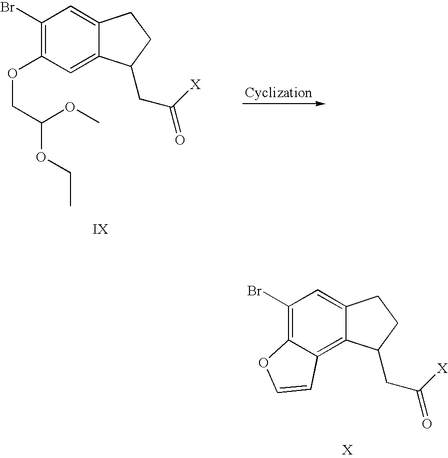

Wherein X=O-Alkyl or —NH₂. In one embodiment X is ethoxy.

The cyclization under heating is conducted in either the absence of a solvent or the presence of a solvent inert to the reaction. Solvents used in this reaction include high-boiling point hydrocarbons such as 1,2,3,4-tetrahydronaphthalen, bromobenzene etc.; high boiling point ethers such as diphenyl ether, dimethyleneglycol dimethyl ether etc., N,N-dimethylaniline, N,N-diethylaniline etc., or a suitable mixture of these solvents are preferable. The reaction is conducted at a temperature of about 0° C. to about 250° C.; preferably 10-90° C. The reaction time is generally 1 hr to 10 hr; preferably 1 hr to 8 hr and most preferably 6 to 8 hr.

The cyclization under acidic conditions uses the acidic substances such as phosphous oxychloride, phosphorus pentoxide, thionyl chloride, hydrobromic acid, hydrochloric acid, sulfuric acid, phosphoric acid, polyphosphoric acid, p-toluenesulfonic acid, methane sulphonic acid, trihaloacetic acid; preferably methane sulfonic acid, trifluoroacetic acid, p-toluene sulphonic acid. Typically, the molar amount of acidic substance is 0.3 to 10 times the molar amount of the compound of formula IX; preferably 0.3 to 2 times. The reaction may be conducted in a solvent inert to the reaction or without a solvent. The reaction temperature is generally 10-150° C., preferably 10 to 50° C. The solvents of the reaction include C₆₋₁₂ aromatic hydrocarbons, C₄₋₇ saturated hydrocarbons, ethers such as tetrahydrofuran, dioxane, 1,2-dimethoxyethane; amides such as N,N-dimethylformamide, N,N-dimethylacetamide; halogenated hydrocarbons such as dichloromethane, dichloroethane, chloroform, carbon tetrachloride; anhydrides such as acetic anhydride; sulfoxides, such as dimethylsulfoxide; water; or mixture thereof. The reaction time is generally 1 hr to 9 hr, preferably 2 hr to 8 hr.

In the case where the cyclization is conducted by using a basic substance, the basic substance includes, for example, sodium hydroxide, potassium hydroxide, sodium carbonate, potassium carbonate, sodium hydrogencarbonate. The basic substance is used in an amount of approximately 0.4 to 10 moles, preferably approximately 5.0 to 20 moles per mol of compound formula VIII. The reaction may be conducted in a solvent inert to the reaction or without a solvent. The solvent of the reaction includes alcohols such as methanol, ethanol, propanol, etc.; ketones such as acetone, methyl ethyl ketone; water; or a suitable mixture of these solvents. The reaction time is generally 30 minutes to 10 hours, preferably 30 minutes to 6 hours. The reaction temperature is generally 20-150° C.; preferably 20 to 100° C.

The compound of Formula X can then be used to prepare Ramelteon.

In another embodiment, the present invention encompasses a process for preparing the Ramelteon intermediate of formula XI, comprising dehalogenating the compound of formula X by using metal hydrides such as NaBH₄ or LiLH₄, or catalytic reduction in presence of Pd—C, Raney-Ni, Zn/HCl, Fe/HCl, hydrogen atmosphere 0.1 kg to 100 kg pressure, preferably 5-10 kg pressure. The preferable reduction catalysts are Pd—C, Raney-Nickel, Zn/HCl and Fe/HCl. The reaction is conducted in a solvent selected from the group comprising of halogenated hydrocarbons, a C₆ to C₁₄ aromatic hydrocarbon, a C₁ to C₅ alcohol, a C₂ to C₇ ester, and a C₂ to C₇ ether, a C₁ to C₅ carboxylic acid, water, or a suitable mixture of these solvents; preferably methanol, isopropyl alcohol, dichloromethane, toluene, ethyl acetate, diethyl ether. The reaction temperature is generally 15-100° C., preferably 20-40° C. The reaction time is generally 1 hr to 8 hrs, preferably 2 hrs to 4 hrs. Typically, the amount of catalyst used is 2-30 g per 100 g of the compound of formula X; preferably 5-20 g per 100 g of the compound of formula X.

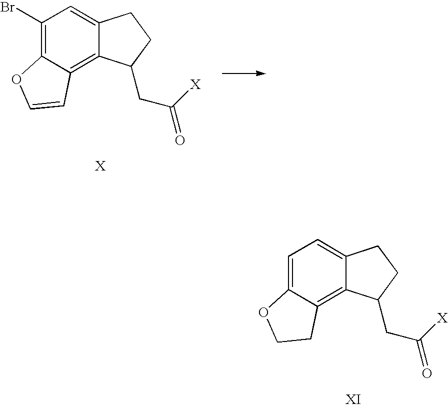

Wherein X=O-Alkyl or —NH₂. In one embodiment X is ethoxy.

The compound of Formula XI can then be used to prepare Ramelteon.

In another embodiment, the present invention encompasses a process for preparing a Ramelteon intermediate of formula XII, comprising reaction of compound of formula XI with aqueous ammonia, or ammonia in any form, preferably in amounts ranging from 1 to 50 moles relative to compound of formula X, more preferably 2-20 moles. The reaction is carried out in the presence of a solvent selected from halogenated hydrocarbons, C₆ to C₁₄ aromatic hydrocarbon, C₁ to C₅ alcohol, C₂ to C₇ ester, and C₂ to C₇ ether or mixtures thereof. Preferably the solvent is methanol, isopropanol, ethyl acetate, dichloromethane, in amounts ranging from 1 to 5 volumes relative to compound of formula XI at a temperature ranging from 20 to 150° C., preferably from 20 to 50° C. The reaction time is usually about 1 hr to about 10 hr; preferably about 3 hr to about 6 hr.

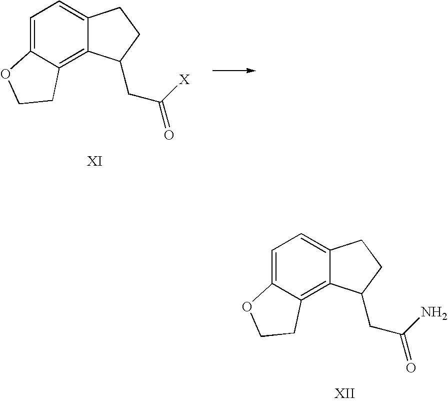

Wherein X=O-Alkyl or —NH₂. In one embodiment X is ethoxy.

The compound of Formula XII can then be used to prepare Ramelteon.

In another embodiment, the present invention encompasses a process for preparing the Ramelteon intermediate of formula XIII, as described by Toru Yamano et al, Tetrahedron: Asymmetry 17 (2006) 184-190 & JP 11080106; which comprising reduction of compound of formula XII by adding boron-trifluoride diethyl ether complex and tetrahydrofuran to sodium borohydride at −10° C. The mixture is stirred at room temperature for 1 hr. After cooling to 0° C., compound of formula XII is added and stir at room temperature for 24 hr.

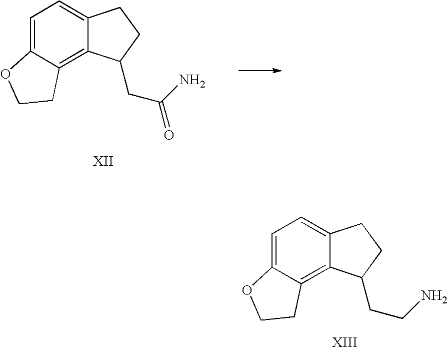

The compound of Formula XIII can then be used to prepare Ramelteon.

In another embodiment, the present invention encompasses a process for preparing Ramelteon of formula I, as described by Toru Yamano et al, JP 11080106; which comprising reacting compound of formula XIII with propionyl chloride in presence of triethyl amine and tetrahydrofuran as solvent.

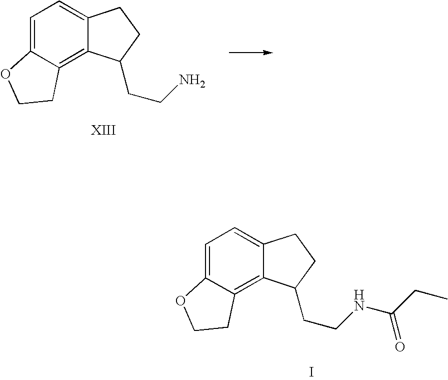

The above steps can be combined to obtain a continuous process starting from intermediates II and III, and ending in compound I. Alternatively, one of ordinarily skill of art could utilize only select steps of this process and combine these steps with other methods. This process, when utilizing all of the steps, comprises preparing Ramelteon, having the Formula I

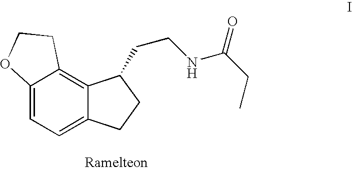

Comprising
(a) reacting the compound of formula II with compound of formula III in presence of base and an organic solvent;

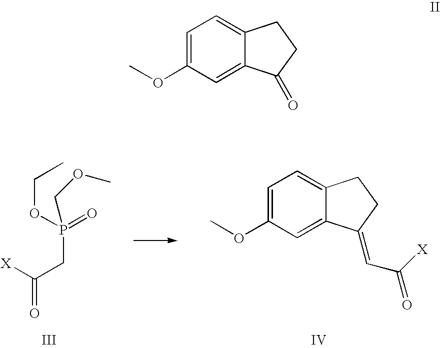

(b) chiral reduction of compound of formula IV in presence Ru-BINAP complex;

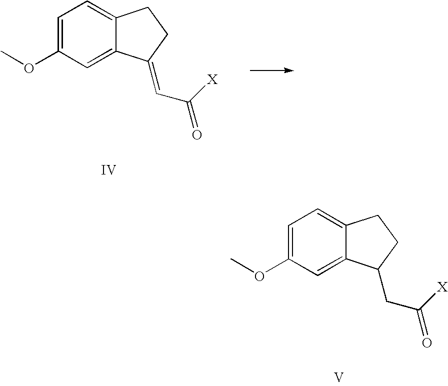

(c) reacting the compound of formula V with brominating agent in presence of an acid or an alkaline salt of organic acid or any acid acceptor;

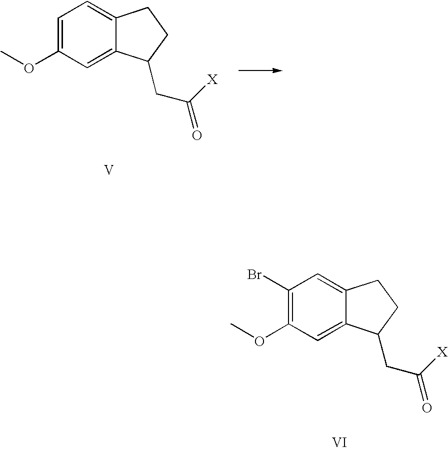

(d) removing the protective group for the hydroxyl group in compound of formula VI;

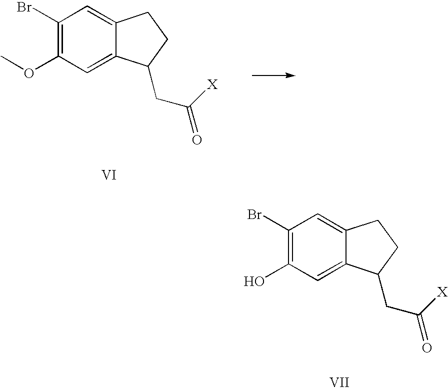

(e) reacting the compound formula VII with the compound of formula VIII in presence of a base to produce the compound of formula IX;

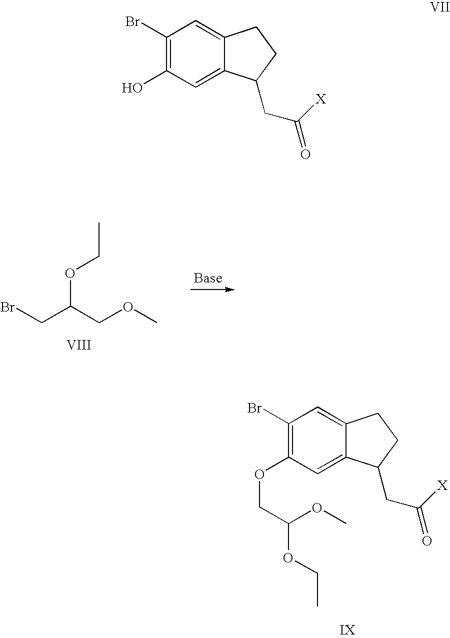

(f) cyclizing the cyclizing the compound of formula IX to produce compound of formula X;

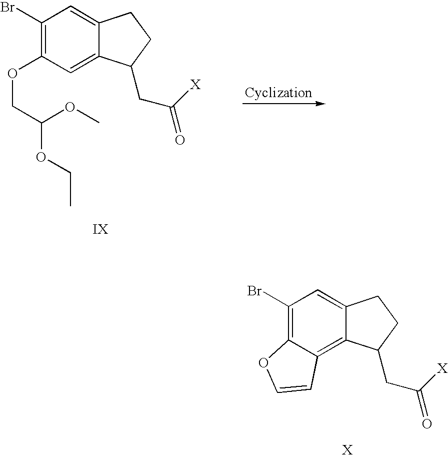

(g) dehalogenating the compound of formula X to obtain compound of formula XI;

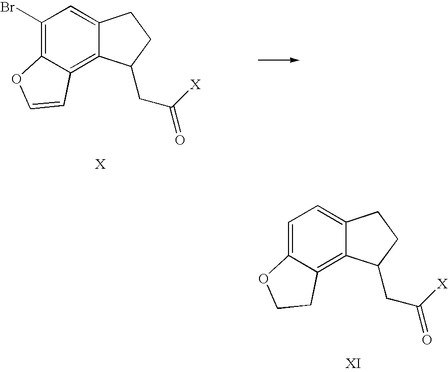

(h) reacting compound of formula XI with aqueous ammonia, or ammonia to obtain compound XII;

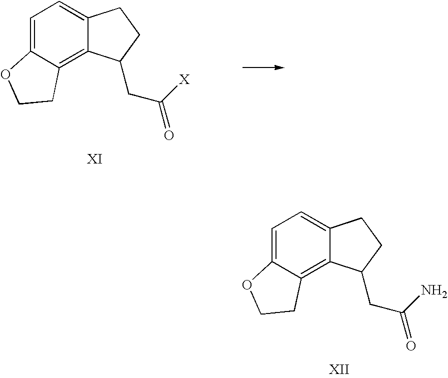

(i) reducing the compound of formula XII to obtain the compound of formula XIII;

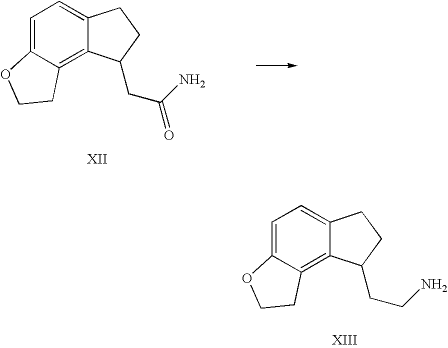

(j) converting compound XIII to compound I;

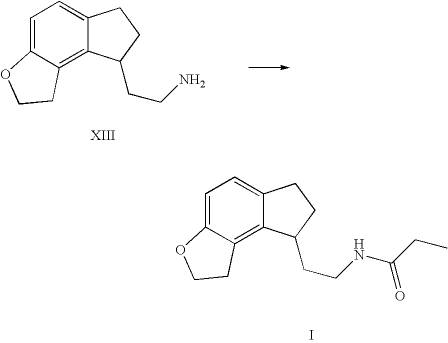

Wherein X=O-Alkyl or —NH₂. In one embodiment X is ethoxy. Detailed conditions for each of these steps (a-j) are provided above.

The present invention provides a compound having the following structure:

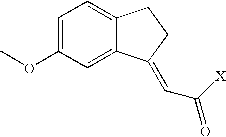

In one embodiment compound IV has the S isomeric structure. Compound IV can be obtained as a product by reacting compounds II and III as described above. After formation of compound IV, water can be added to the reaction

### Compound IV

mixture to obtain two phases, particularly if the reaction is carried out with a water immiscible solvent. The organic layer can be washed. The product can be recovered from the organic layer, such by applying a pressure of less than one atmosphere and/or a heated temperature of about 40 to about 60° C. The product may be purified by HPLC. The product may be purified to obtain a purity of about 50% to about 98%, such as of about 95% to about 98%, as measured by area percentage HPLC.
The present invention provides a compound having the following structure:

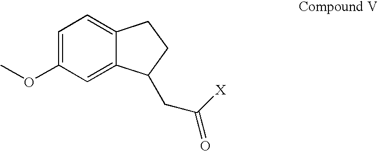

In one embodiment compound V has the S isomeric structure. Compound V can be obtained from compound IV as described above. The reaction mixture can be filtered to remove impurities. Compound V can be recovered from the reaction mixture by evaporating the reaction mixture, such as by applying a pressure of less than one atmosphere and/or a heated temperature of about 40 to about 60° C. The product may be purified to obtain a purity of about 50% to about 98%, such as of about 95% to about 98%, as measured by area percentage HPLC.

The present invention provides a compound having the following structure:

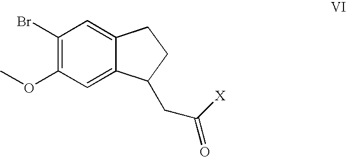

In one embodiment compound VI has the S isomeric structure. Compound VI can be prepared from compound V as described above. It can be recovered from the reaction mixture. During the reaction, the mixture can be stirred to accelerate the reaction between compounds IV and V. Compound VI can be recovered from the organic layer by evaporation, such as by applying a pressure of less than one atmosphere and/or a heated temperature of about 40 to about 60° C. The product may be purified to obtain a purity of about 50% to about 95%, such as of about 90% to about 95%, as measured by area percentage HPLC.

The present invention provides a compound having the following structure:

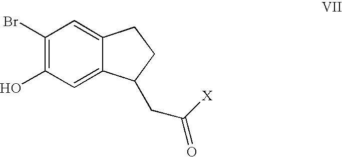

In one embodiment compound VII has the S isomeric structure. Compound VII can be prepared from compound VI as described above. After completion of the reaction, water can be combined with the reaction mixture, to obtain two phases, particularly if the reaction is carried out in a water immiscible solvent. It can be recovered by evaporating the reaction mixture, such as by applying a pressure of less than one atmosphere and/or a heated temperature of about 40 to about 60° C. The product can may purified to obtain a purity of about 50% to about 95%, such as of about 90% to about 95%, as measured by area percentage HPLC.

The present invention provides a compound having the following structure:

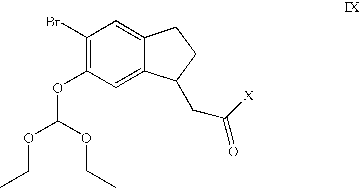

In one embodiment compound IX has the S isomeric structure. Compound IX can be prepared by reacting compounds VIII and VII as described above. After completion of the reaction, water can be combined with the reaction mixture, to obtain two phases, particularly if the reaction is carried out in a water immiscible solvent. It can be recovered by evaporating the reaction mixture, such as by applying a pressure of less than one atmosphere and/or a heated temperature of about 40 to about 60° C. The product may be purified to obtain a purity of about 50% to about 95%, such as of about 90% to about 95%, as measured by area percentage HPLC.

The Ramelteon prepared by the process of the invention may be used for treatment of insomnia. It can be combined with a pharmaceutically acceptable excipient to prepare pharmaceutical compositions.

Having described the invention with reference to certain preferred embodiments, other embodiments will become apparent to one skilled in the art from consideration of the specification. The invention is further defined by reference to the following examples describing in detail the process and compositions of the invention. It will be apparent to those skilled in the art that many modifications, both to materials and methods, may be practiced without departing from the scope of the invention.

### EXAMPLES

All purity described in the examples is determined by HPLC.

### Synthesis of Intermediate-IV

### Example I

A compound of formula IV was prepared by Witting-Horner reaction in which compound of formula II (100.0 gm, 0.6165 mol) was reacted with triethyl phosphono acetate (276.21 gm, 1.2331 mol) in presence of 60% NaH (29.5 gm, 1.2331 mol) in toluene (1000 ml) under nitrogen atmosphere. Reaction was stirred for 18-24 hr at 85-95° C. under nitrogen atmosphere. Reaction progress was checked by TLC and HPLC. The reaction mixture was cooled to room temperature (RT) and water was added into it after completion of reaction. The organic layer and aqueous layer were separated. The aqueous layer was extracted with toluene and then combined organic layer was washed with 10% brine solution. Distilled out the organic layer under vacuum at 45-50° C.

Yield: 65-70% Purity 92-95%.

NMR of compound IV

1.178-1.218 (t, 3H), 3.294 (s, 2H), 3.613 (s, 2H), 3.759 (s, 3H), 4.070-4.141 (q, 2H), 6.462 (s, 1H), 6.754-6.790 (dd, 1H) 6.920-9.928 (s, 1H), 7.330-7.357 (d, 1H).

### Synthesis of Intermediate-V

### Example 2A

Compound of formula IV (100.0 gm, 0.4305 mol) was reduced in methanol (1500.0 ml) and water (300.0 ml) mixture in presence of 5% Pd/C (13.0 gm) in hydrogen pressure. The reaction mixture was stirred for 2-3 hrs at RT and reaction progress was monitored by HPLC and TLC. The reaction mixture was filter on hyflow bed after completion of reaction. Distilled out solvent under reduce pressure at 45-50° C.

Yield: 92-95% Purity 92-95. %

NMR of compound V—1.178-1.218 (t, 3H), 3.294 (s, 2H), 3.613 (s, 2H), 3.759 (s, 3H), 4.070-4.141 (q, 2H), 3.486-3.555 (pentate, 1H), 4.415-4.456 (m, 2H), 6.754-6.790 (dd, 1H) 6.920-9.928 (s, 1H), 7.330-7.357 (d, 1H).

### Example 2B

### Chiral Reduction

Compound of formula IV (100.0 gm, 0.4305 mol) is reduced in methanol (500.0 ml) and toluene (500 ml) mixture in presence of [RuCl(benzene)((R)-BINAP)]Cl, under hydrogen pressure. The reaction mixture is stirred for 20-22 hr at 80-90° C. and reaction progress is monitored by HPLC and TLC. After completion of reaction, the reaction mixture is filtered trough hyflow and Mixture of toluene and methanol is distilled out under reduce pressure at 50-55° C.

### Synthesis of Intermediate-VI

### Example 3

Compound of formula V (100.0 gm, 0.4304 mol) in methanol (1000.0 ml) was stirred in presence of sodium acetate (38.8 gm, 0.4735 mol) for 5-10 minute and then bromine (68.6 gm, 0.4304 mol) was added drop wise into it at 0-5° C. The reaction mixture was stirred for 2-3 hrs at 0-5° C. Sodium bisulphite solution was added after completion of reaction and stir reaction mass for 5-10 min and then distilled out methanol under reduce pressure at 45-50° C. MDC (methylene dichloride) was added after distillation. The organic layer was separated and washed it with 5% sodium bisulphite solution. MDC layer was distilled out under reduce pressure at 40-45° C.

Yield: 90-92%; Purity: 92-97%

NMR of Compound VI

1.258-1.305 (t, 3H), 1.721-1.837 (m, 1H), 2.332-2.460 (m, 2H), 2.676-2.928 (m, 3H), 3.492-3.587 (pentate, 1H), 3.859-3.890 (s, 3H), 4.146-4.217 (q, 2H), 6.763 (s, 1H), 7.376 (s, 1H).

### Synthesis of Intermediate-VII

### Example 4

A mixture of compound of formula VI (100.0 gm, 0.3192 mol) in dichloromethane (1500 ml) was cooled at −25 to −30° C. BBr₃ (159.9 gm, 0.6385 mol) was added dropwise into reaction mixture at −25 to −30° C. The reaction mixture was stirred for 3-4 hrs at −25 to −30° C. Reaction progress was checked by TLC and HPLC. Reaction mixture was poured into chilled water with stirring. The layers were separated. The organic layer was washed with 10% brine solution. Distilled out dichloromethane layer and obtained product as a liquid which was solidified. Yield: 80-85%; Purity by area percentage by HPLC: 95-98%.

NMR of Compound VII

1.259-1.355 (t, 3H), 1.689-1.808 (m, 1H), 2.322-2.455 (m, 2H), 2.672-2.856 (m, 4H), 3.461-3.557 (pentate, 1H), 4.148-4.218 (q, 2H), 6.843 (s, 1H), 7.290 (s, 1H).

### Synthesis of Intermediate-IX

### Example 5

Added Compound VII and 3-5 vol of Dimethyl formamide, 2.5 mole equivalent Sodium Hydride is added at −05 to 15° C. and stirred the reaction for 1-2 hr at −05 to 15°. Add Compound VIII lot wise and heat it to 80-95 temperature stir for 3-5 hrs, after completion of reaction cool the mass to 25-30 temperature, add acetic acid to adjust pH neutral, add Water 30-50 times to isolate product. Filter the slurry. Yield is 80%-85%. Purity 95-99%.

NMR data: 1.203-1.238 (t, 6H), 2.281-2.384 (m, 1H), 2.521-2.573 (q, 1H), 2.742-2.829 (m, 2H), 3.523-3.559 (q, 1H), 3.942-4.012 (m, 2H), 4.816-4.842 (m, 1H), 5.373 (s, 2H), 6.770 (s, 1H), 7.334 (s, 1H).

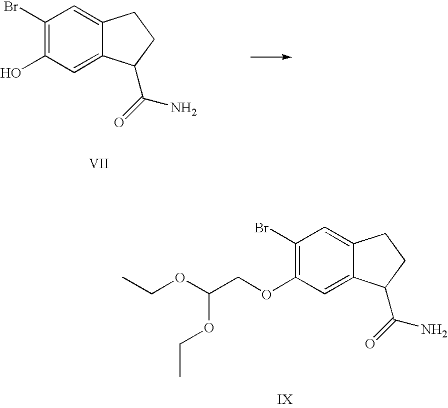

### Synthesis of Intermediate-X

### Example 6

Compound of formula IX (100.0 gm, 0.2407 mol) is added to methane sulfonic acid (46.22, 0.4814 mol) in toluene (1000.0 ml) at 25-35° C. and the reaction mixture is stirred at 25-35° C. for 6-8 hr. Reaction progress is monitored by HPLC and TLC, after completion of reaction, reaction mixture is poured into ice cold water. Separate organic and aqueous layers and organic layer is washed with water and brine solution. Solvent is distilled off from organic layer to obtain the title compound.

### Synthesis of Intermediate-XI

### Example 7

Compound of formula X (100.0 gm, 0.3094) is hydrogenated by 10% Pd/C (15.0 gm) in methanol (2000.0 ml) and water (200.0 ml). Reaction mixture is stirred for 2-3 hrs at 25-30° C. under hydrogen atmosphere 5-10 kg pressure. Reaction is monitored by HPLC and TLC. After completion of reaction, filter the reaction mixture on Hyflow and then solvent is distilled off under reduced pressure at 45-50° C. to obtain the compound of formula XI.

### Synthesis of Intermediate-XII

### Example 8

A mixture of compound of formula XI (100.0 gm, 0.4059) in methanol (100.0 ml) and aqueous ammonia solution (500.0 ml) is stirred under for 5-6 hr at 25-35° C. Progress of reaction is monitored by HPLC and TLC. Reaction mixture is poured into water and then extract twice with ethyl acetate. Organic layer is washed with brine solution and then dried with sodium sulfate and organic layer is distilled off under reduced pressure at 45-50° C. The product is isolated.

### Synthesis of Intermediate-XIII

### Example 9

The boron-trifluoride diethyl ether complex (350 ml, 277 mmol) is added THF 2500 ml) and cool it to −10-15° C. The sodium borohydride (104 gm, 277 mmol) is added to reaction mixture and raise the temperature to 25-35° C. and stir for 1-1.5 hr at 25-35° C. Again this liquid is cooled and compound of formula XII (100.0 gm, 460 mmol) is added. After addition the reaction is stirred at 25-35° C. for 24-25 hr. The mixture is concentrated under reduced pressure to yield solids, which are dissolved in ethyl acetate and treated with 1M hydrochloric acid. The mixture is concentrated to dryness and the resulting residue is washed with diisopropyl ether to afford the hydrochloride salt of compound of formula XIII.

### Synthesis of Ramelteon (I)

### Example 10

The hydrochloride salt of compound of formula XIII (100.0 gm, 418 mmol) is suspended in the THF at 4000 ml, triethyl amine (116.0 ml, 836 mmol) is added and the reaction is cooled to 10° C. or less. Propionyl chloride (74 ml, 836 mmol) is added dropwise followed by agitation at 25-35° C. for 2-3 hrs. Then 1000 ml, of water is added and the THF is distilled off under reduced pressure. It dissolved in ethyl acetate and wash twice with 10% brine solution. Dry the organic layer with sodium sulfate, distill off under vacuum and product is isolated. Dry the product under vacuum.

What is claimed is:

1. A compound having the following structure: 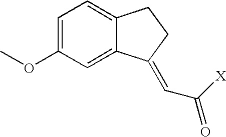
wherein X is O-Alkyl or —NH₂.

2. The compound of claim 1, wherein X is ethoxy.

3. The compound of claim 1, wherein the compound has a purity of at least about 50% as measured by area percentage HPLC.

4. A compound having the following structure: 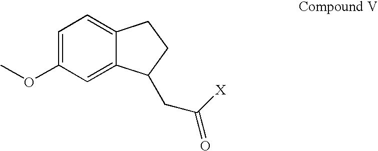
wherein X is O-Alkyl or —NH₂.

5. The compound of claim 4, wherein X is ethoxy.

6. The compound of claim 4, wherein the compound has a purity of at least about 50% as measured by area percentage HPLC.

7. The compound of claim 4, wherein the compound has (s) isomeric configuration.

8. A compound having the following structure: 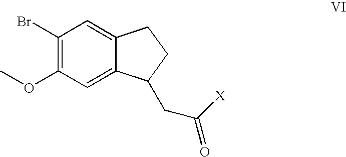
Wherein X=O-Alkyl or —NH₂.

9. The compound of claim 8, wherein X is ethoxy.

10. The compound of claim 8, wherein the compound has (s) isomeric configuration.

11. The compound of claim 8, wherein the compound has a purity of at least about 50% as measured by area percentage HPLC.

12. A compound having the following structure: 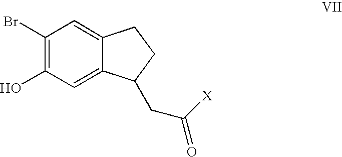
Wherein X=O-Alkyl or —NH₂.

13. The compound of claim 12, wherein X is ethoxy.

14. The compound of claim 12, wherein the compound has (s) isomeric configuration.

15. The compound of claim 12, wherein the compound has a purity of at least about 50% as measured by area percentage HPLC.

16. A compound having the following structure 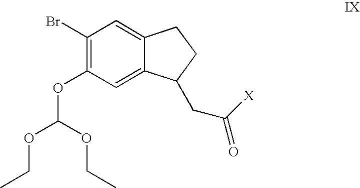
wherein X is O-Alkyl or —NH₂.

17. The compound of claim 16, wherein X is ethoxy.

18. The compound of claim 16, wherein the compound has (s) isomeric configuration.

19. The compound of claim 16, wherein the compound has a purity of at least about 50% as measured by area percentage HPLC.

20. A process for producing Ramelteon intermediate of formula IV comprising combining compound of formula II with compound of formula III in the presence of base and organic solvent. 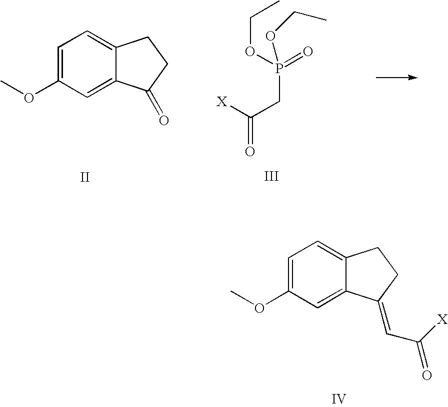
Wherein X=O-Alkyl or —NH₂.

21. The process of claim 20, wherein X is ethoxy.

22. A process for preparing Ramelteon comprising preparing the compound of Formula IV according to claim 20, and converting it to Ramelteon.

23. A process for preparing a Ramelteon intermediate of formula V, comprising reduction of compound of formula IV in presence of Ru-BINAP complex under hydrogen atmosphere in an organic solvent. 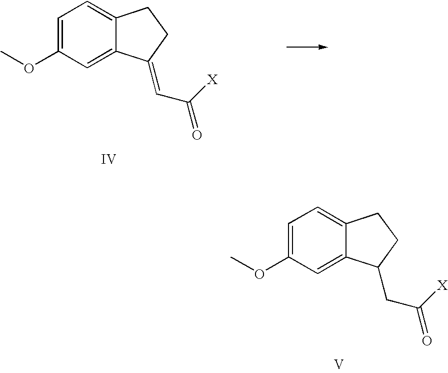
Wherein X=O-Alkyl or —NH₂.

24. The process of claim 23, wherein X is ethoxy.

25. A process for preparing Ramelteon comprising preparing compound of formula V according to claim 23, and converting it to Ramelteon.

26. A process for preparing a Ramelteon intermediate of formula VI, comprising reacting compound of formula V with brominating agent in presence of an acid or alkaline salt of an acid. 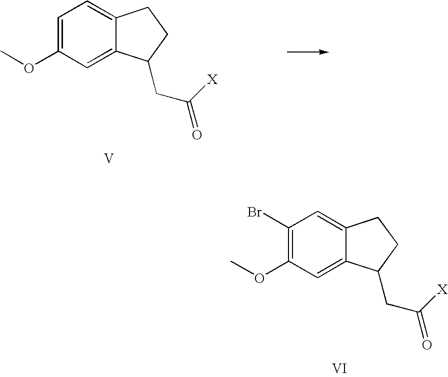
Wherein X=O-Alkyl or —NH₂.

27. The process of claim 26, wherein X is ethoxy.

28. A process for preparing Ramelteon comprising preparing compound of Formula VI as described in claim 26 and converting it to Ramelteon.

29. A process for preparing a Ramelteon intermediate of formula VII, comprising removing protective group for the hydroxyl group in compound of formula VI. 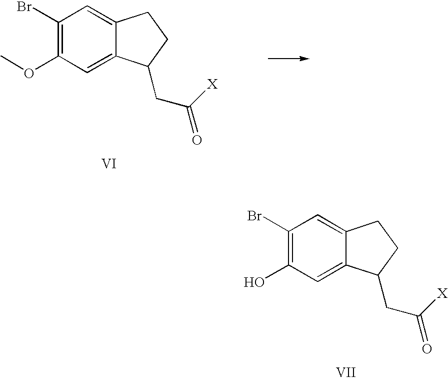
Wherein X=O-Alkyl or —NH₂.

30. The process of claim 29, wherein X is ethoxy.

31. A process for preparing Ramelteon, comprising preparing compound of formula VII as described in claim 29, and converting it to Ramelteon.

32. A process for preparing the Ramelteon intermediate of formula IX, comprising reacting compound of formula VII with compound of formula VIII in presence of a base to produce the compound of formula IX: 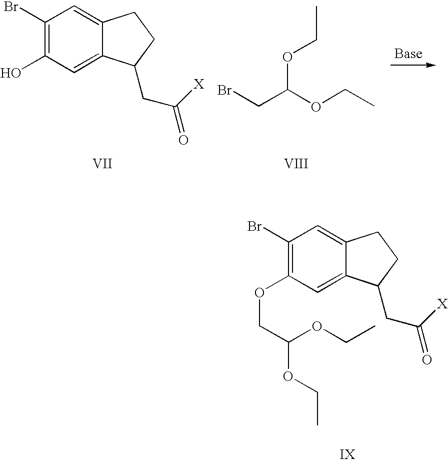
Wherein X=O-Alkyl or —NH₂.

33. The process of claim 32, wherein X is ethoxy.

34. A process for preparing Ramelteon comprising preparing the compound of Formula IX as described in claim 32, and converting it to Ramelteon.

35. A process for preparing the compound of formula IX Comprising:
a) reacting the compound of formula II with compound of formula III to obtain compound of formula IV: 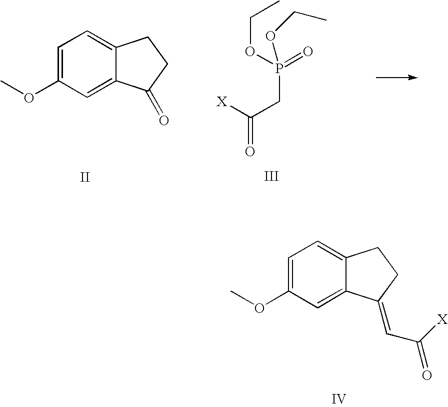
b) reduction of compound of formula IV to obtain compound of formula V: 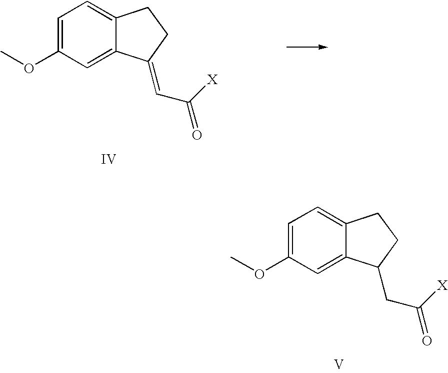
c) combining compound of formula V with brominating agent to obtain compound of formula VI: 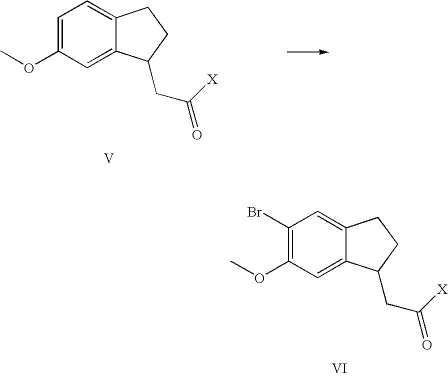
d) demethylation of the compound of formula VI to obtain the compound of formula VII: 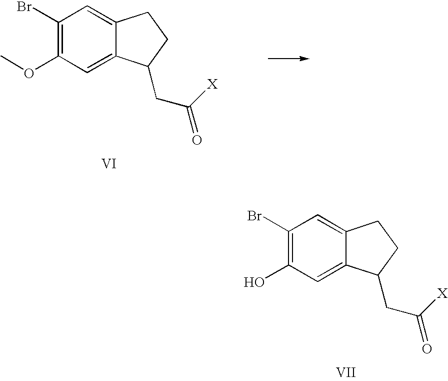
e) combining the compound of formula VII with compound of formula VIII in presence of base to produce the compound of formula IX: 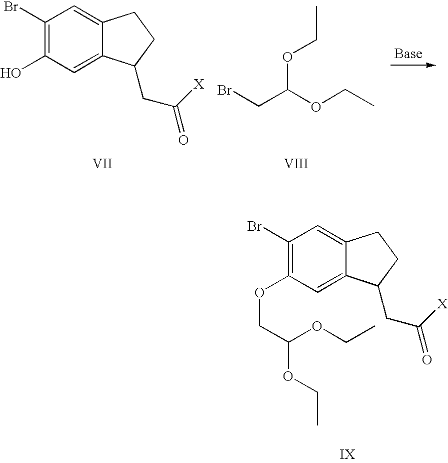
f) converting compound IX into Ramelteon;
Wherein X=O-Alkyl or —NH₂.
